(19) United States
(12) Patent Application Publication
(10) Pub. No.: US 2005/0038362 A1
(43) Pub. Date: Feb. 17, 2005

(54) Device for generation of different pressure waves by means of variable reflector areas

(75) Inventor: Reiner Schultheiss, Illighausen (CH)

Correspondence Address: JOYCE VON NATZMER, 4615 NORTH PARK AVENUE, SUITE 919, CHEVY CHASE, MD, 20815, US

(73) Assignee: SWS Shock Wave Systems AG, Illighausen (CH)

(21) Appl. No.: 10/754,586

(22) Filed: Jan. 12, 2004

(30) Foreign Application Priority Data
Jan. 17, 2003 (DE) 10301875

(51) Int. Cl. H04R 1/00; A61B 17/22

(52) U.S. Cl. 601/4; 367/147; 367/174

(57) ABSTRACT

The invention relates to an apparatus for the generation of shockwaves, consisting of a pressure pulse source (7), at least one reflector element (2, 3, 102, 103, 104), a shockwave permeable diaphragm (8) and a shockwave conducting medium and enables the generation of different shockwave profiles in the treatment area without extensive modification or remounting.

For this purpose the apparatus is equipped with an adjustment mechanism for changing the size of the reflection surface (5) located within the radiation path of the primary pressure pulse.

The invention relates to an apparatus for the generation of shockwaves, consisting of a pressure pulse source, at least one reflector element, a shockwave permeable membrane and a shockwave conducting medium.

### PRIOR ART

Acoustic shockwaves, i.e. pressure pulses with a steep leading edge, a high peak amplitude and a short pulse duration, are used in medicine to smash stones, to stimulate bone growth, to treat joint disorders, to stimulate nerves, to improve circulation, to stimulate the growth of new blood vessels, to treat pain and to narcotize certain bodily areas, whereby this only represents a selection of the possible areas of application.

Various methods are known for generating shockwaves, for example by means of piezo-electric systems, electro-magnetic systems and electro-hydraulic systems. In most cases a primary pressure pulse is generated, which impacts a reflector through a shockwave conducting medium and is then bundled to a focal volume. The increasing shockwave pulse passes through a diaphragm closing off the reflector and is then coupled into the treatment area, i.e. the bodily area to be treated.

This creates a shockwave profile in the treatment area that can be characterized by the temporal curve of the pressure parts and the rarefaction wave parts, by the spatial distribution of the maximum amplitudes, the diffraction structures, the energy flow densities and the overall transferred energy.

Such an apparatus is described, for example, in DE 197 18 513. In this publication, the reflector surface is a partial ellipsoid that is rotationally symmetric relative to the exit axis, whereby in the first geometric focus an electro-hydraulic shockwave is generated and is reproduced on the second geometric focus located outside of the apparatus. The elliptic geometry causes parts of the shockwave to arrive in the focal volume at the same time.

A further publication, EP 0 189 756, describes that the primary pressure pulse is generated as an even wave by a shock tube, and this wave passes over a dividing apparatus, from which it impacts two reflectors with different geometries, of which they are directed at the same focal volume. The parts of the shockwaves that pass over the respective reflector surfaces arrive in the focal area at staggered intervals. The time difference can be varied by using different reflectors and distances between the pressure pulse generator and the reflectors.

Furthermore, a pressure pulse generator is known from DE 41 22 590 in which the focusing means is adjustable relative to the pressure pulse source, so that defocusing is possible.

### DISADVANTAGES OF THE PRIOR ART

The known shockwave generation systems, which direct the shockwave by means of reflection, are not adequately able to generate a changeable shockwave profile in the treatment area.

The peak amplitude in the treatment area can be influenced by the voltage applied at the pressure pulse source. The combination of applied voltage and reflector geometry, however, defines the shockwave profile in the target area. Only by changing the penetration depth, i.e. the distance between the coupling surface and the focal volume, can the relative position of the treatment area and the fixed shockwave profile be selected. Different penetration depths are achieved by coupler pads or by a variable quantity of the shockwave conducting medium and the corresponding arching of the diaphragm. Changing the penetration depth requires extensive adjustment processes, such as replacing the treatment heads or re-pumping the shockwave conducting medium.

### OBJECT OF THE INVENTION

The object of the invention is to present an apparatus with which different shockwave profiles can be generated in the treatment area without extensive modification or remounting.

### ACHIEVEMENT OF THE OBJECT

The object is achieved by equipping the apparatus with an adjustment mechanism for changing the size of the reflection surface located in the radiation path of the primary pressure pulse.

### ADVANTAGES OF THE INVENTION

The shockwave profile is affected substantially by the reflector geometry. This is especially clear in electro-hydraulic shockwave generation, in which as a rule partial ellipsoids are used as reflector elements. The long semi-axis is identical to the exit axis.

Depending on the absolute dimensions and the relationship of the semi-axes, the distance of the focal points and thus the position of the focus area changes relative to the location of the shockwave pulse generation. Once the semi-axes have been defined, the actual elliptic section used also affects the shockwave profile. The size of the exit surface and the aperture angle depends on the section of the long semi-axis where the rotational ellipsoid is cut. This is the angle located between two imaginary opposing straight lines, which extend from the focal point outside of the reflector to the edges of the reflector. The longer the reflector, the more obtuse is the aperture angle; a flat cut ellipsoid produces an acute aperture angle, since the distance from the edge of the reflector to the focal point is larger. At the same time, a flat cut ellipsoid enables a greater distance between the reflector edge and the focal zone, and vice versa, i.e. a long reflector enables a smaller distance between the reflector edge and the focal zone. In short, this produces the following relationship: a large aperture angle corresponds to a low penetration depth and vice versa.

The aperture angle is also a measure for the distortion of the shockwave profile, because the diffraction pattern is affected primarily by the relative angle of the converging wave sections. Due to non-linear effects, the shockwave does not satisfy simple laws of radiation and no precise reproduction of the partial cone-shaped primary shockwave pulse is generated in the focal area. Reflectors with different apertures cause focal volumes with different extents, since in a first approximation the reflector geometry and, due to the diffraction pattern, the aperture is also reproduced.

In the case of a reflector with a rotationally symmetrical reflection surface, the reflection surface is larger in relation to the depth of the reflector, and in the case of an ellipsoid, the nearer the length of the cut is to the large semi-axis. The reflection surface in turn is a measure for the intensity of the shockwave, since the larger the solid angle covered by the surface, the more parts of the primary shockwave will be directed into the focal volume and the less parts of the primary shockwave pulse will be undirected and lost.

The apparatus according to the invention makes it possible to influence the aperture angle and/or the pressure curve in the focal volume, without changing the entire reflector geometry, in particular favorable axial ratios, by adding and removing surface elements to/from the reflector surface. Surface elements of the reflector can be covered, replaced, pushed away or folded back, which can be achieved by means of a switch, lever or slide element.

In a preferable embodiment of the invention, the pressure pulse source and the reflection surface are located in a housing, which is sealed on one side with the pressure pulse permeable diaphragm. In this case, the diaphragm is not connected directly with the reflector, allowing more freedom in the arrangement of the elements. The housing and the diaphragm enclose the shockwave conducting medium, so that the reflector does not serve as a primary container for this medium, and in particular must not have a sealing function.

In an especially advantageous embodiment of the invention, the pressure pulse source and/or at least one reflector element and/or parts thereof and/or the diaphragm surface are movable in relation to the housing. Preferably the pressure pulse source always remains in a particular position in relation to the reflector geometry, e.g. in the first focal point of an ellipsoid. If the reflector is now moved relative to the diaphragm, the penetration depth can be varied, for example.

If only the reflector or the pressure pulse source is moved within the housing, then this changes the relative position of the pressure pulse source and of the reflector to each other and the entire apparatus can be defocused, for example.

In many apparatuses known in the art with a pre-defined reflector surface, the penetration depth is changed by pumping up the diaphragm. This procedure is possible also with the apparatus according to the invention.

In a preferred embodiment, the apparatus consists of at least two reflector elements, of which at least one is movable relative to the other and/or relative to the pressure pulse source and/or relative to the diaphragm. Upon a relative movement of reflector elements to each other, the reflector surface can be changed by pushing one reflector element behind another, so that it is no longer in the radiation path of the primary shockwave pulse. A reflector element with certain reflection properties can also be pushed in front of another reflector element, thus reducing its reflection surface.

In an advantageous further embodiment of the invention, the inner surfaces of the reflector elements have a reflection surface that is rotationally symmetrical relative to the exit axis and at least one reflector element can be moved axially by sliding out of and into the radiation path of the pressure pulses. At least one reflector element in this case has a ring structure, and this ring can be moved in the direction of the symmetry axis, which corresponds to the exit axis.

This design can be useful if the reflector breaks down into two parts. The outer reflector ring can be pushed back and forth along the exit axis; when pushed forward it enlarges the reflector surface and the aperture angle. When pushed back, the ring-shaped reflector element does not contribute to the reflection, since it is not located in the radiation path of the primary pressure pulse. The reflector surface then consists only of the ellipsoidal reflector body.

This basic structure can be achieved with any number of movable reflector rings. Preferably the ring width is such that the rings of a given material thickness fit into each other. If the diaphragm is coupled to the outermost ring, then moving the rings changes not only the reflector surface, but also the penetration depth, since the distance of the pressure pulse source relative to the exit surface defined by the diaphragm changes.

In a further preferred embodiment, the inner surfaces of the reflector elements can be connected to a rotationally symmetric reflection surface relative to the exit axis and a part of the reflector elements can turn on bearings on an axis that can be parallel or perpendicular to the exit axis, so that these reflector elements are hinged for moving into and out of the radiation path of the pressure pulse. This apparatus can be used to change the reflector surface, without affecting the aperture angle.

This can also be achieved if the inner surfaces of the reflector elements can be connected to a reflection surface relative that is rotationally symmetrical to the exit axis and some of the reflector elements can rotate on the exit axis, so that they can be moved parallel to the reflector elements that are stationary.

In both embodiments the exit surface and thus the aperture angle remain unchanged when the size of the reflector surface is changed. Therefore, the intensity of the shockwave can be influenced directly, without changing the electric parameters of the pressure pulse source. In this way, a device for shockwave generation with adjustable shockwave parameters can be operated and at the same time the generally complex pressure pulse generation source can be maintained at constant electromagnetic conditions.

The adjusting mechanism is preferably designed so that the movement of the movable elements can be achieved manually, electrically, hydraulically or pneumatically.

In preferable embodiments, the reflector elements making up the reflection surface have different reflection coefficients. They are determined by the propagation times of the shockwave in the material and the damping constants. The acoustic parameters can be defined by differing wall thicknesses, surface roughnesses, materials or other physical parameters of the reflector elements.

For example, with the same thickness, the density (p=8900 kg/m³) and acoustic velocity (c=4660 m/s) of copper produce a different acoustic impedance (p*c) and therefore different reflection properties than zinc (p=7100 kg/m³, c=4170 m/s) or various ceramics (e.g. Borgias with p=1800 kg/m³ and c=3470 m/s, or quartz glass with p=2200 kg/m³ and c=5370 m/s) or plastics (e.g. polyethylene with p=960 kg/m³ and c=2500 m/s, or rubber with p=1300 kg/M³ and c=1400 m/s).

In a preferred embodiment of the apparatus, the reflector elements connect to form one reflection surface, which focuses the shockwaves on a focal volume element located outside of the apparatus.

In a further preferred embodiment, a measuring means is attached to the apparatus that indicates where the focal volume element is located. This can be achieved in the form of a mechanical indicator.

Preferably, means are attached to the apparatus that indicate the position of the reflector elements. This indicator provides a measure for the size of the reflector surface and for the quality of the shockwave. The position of the reflector elements can be measured by the mechanical fit, with an electric switch, inductively, capacitively, optically or otherwise. Preferably the measured values are recorded by a control or analysis unit.

Variations in the focal volume and the penetration depth enable the use of a single shockwave source for different areas of application, for example smashing concrements of different sizes in different bodily areas and additionally for the treatment of soft tissue and bone disorders.

Further preferred embodiments will be apparent from the following description and from the claims.

### DRAWINGS

The invention is illustrated in drawings, where:

FIG. 1 shows a section through an apparatus according to the invention with two reflector elements, in a first position;

FIG. 2 shows a section through an apparatus according to the invention with two reflector elements, in a second position;

FIG. 3 shows a section through an apparatus according to the invention with two reflector elements, in a third position;

FIG. 4 shows a top view of a reflector consisting of a plurality of reflector elements;

FIG. 5 shows a side view of a reflector consisting of a plurality of reflector elements.

### DESCRIPTION OF THE EXEMPLARY EMBODIMENTS

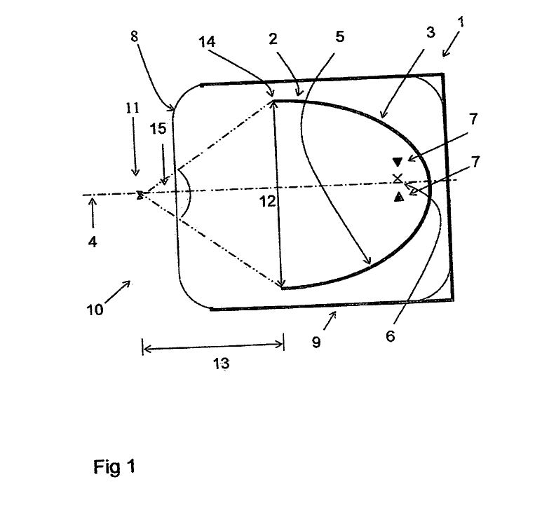

FIG. 1 shows a section through an apparatus 1 according to the invention with two reflector elements 2,3, in a first position. The reflector elements 2,3 are segments of an ellipsoid not explicitly depicted in the drawing and which is rotationally symmetrical on an exit axis 4 and form together a maximum reflection surface 5 in the position depicted here. In a focal point 6 of the ellipse a primary pressure pulse is generated by the pressure pulse source 7 schematically indicated in the drawing. This primary pressure pulse is reflected on the reflection surface 5 and exits the apparatus 1 through the diaphragm 8, which closes the housing 9 toward the exit side 10. The pressure pulses are focused in the second focal point 11. The apparatus 1 must therefore be positioned relative to the patient so that the focal point 11 is located within the desired treatment area. The aperture angle 15 results from the width of the reflector aperture 12 and the distance 13 of the focal point 11 from the edge of the reflector 14.

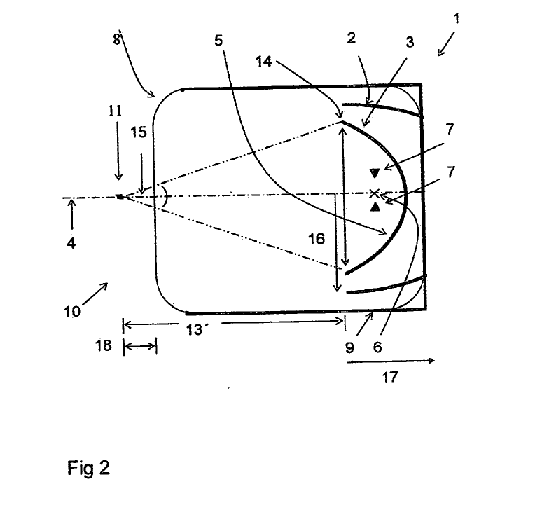

The apparatus 1 according to the invention can be operated as shown in FIG. 2 with a reflection surface 5′, which is smaller than that shown in FIG. 1. For this purpose the reflector element 2 is moved with the larger radius 16 along the exit axis in the direction 17 of the pressure pulse source 7. The reflector element 2 is then no longer located within the radiation path of the primary pressure pulse and therefore does not contribute to the focusing of the pressure pulse. The pressure pulse is then only focused by the reflection surface 5′ of the other reflector element 3. In this position of the reflector elements 2,3 the distance 13′ between the second focal point 11 and the edge of the reflector relative to the position shown in FIG. 1 is enlarged and the aperture angle 15′ therefore becomes more acute.

The penetration depth 18, which determines the distance of the second focus 11 from the diaphragm 8, remains unchanged relative to the position of the apparatus 1, as shown in FIG. 1.

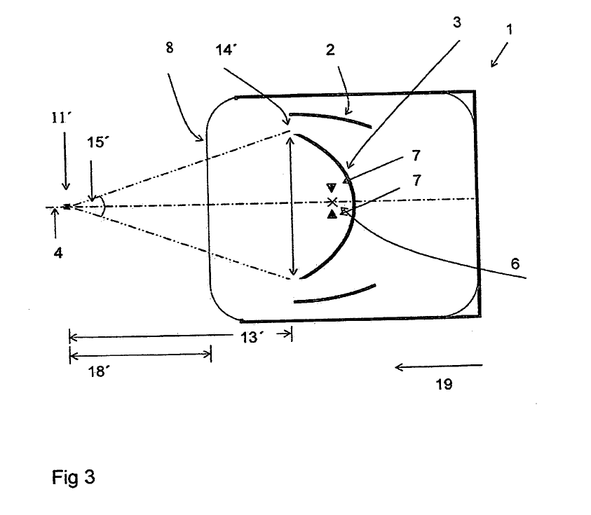

The reflector elements 2,3 of the apparatus according to the invention can, however, be moved into a third position, as shown in FIG. 3. Here the reflector element 3 is moved together with the pressure pulse source 7, which still generates a pressure pulse in a focal point 6, along the exit axis 4 in the direction 19 of the diaphragm 8. The reflector element 3 is then located within the reflector element 2, which does not contribute to the focusing of the pressure pulse. The movement of the reflector element 1 causes the penetration depth 18′ to change, i.e. the distance between the second focal point 11′ and the diaphragm 8, whereas the distance 13′ between the focal point 11′ and the edge of the reflector 14′ and therefore also the aperture angle 15′ remains the same as in the position shown in FIG. 2.

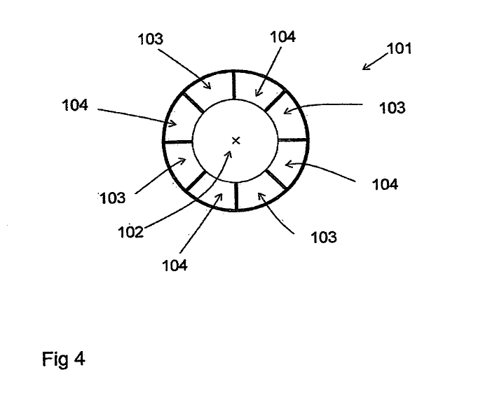

FIG. 4 shows a top view of a reflector 101 consisting of a plurality of reflector elements 102, 103, 104. Within the central reflector element 102 there are pressure pulse sources not depicted in this drawing. The central reflector element 102 is surrounded by stationary reflector elements 103 and by movable reflector elements 104. The movable reflector elements 104 can be pushed behind the stationary reflector elements 103. They are then no longer located within the radiation path of the primary pressure pulse and then only the surfaces of the central reflector elements 102 and of the stationary reflector elements 103 make up the reflection surface.

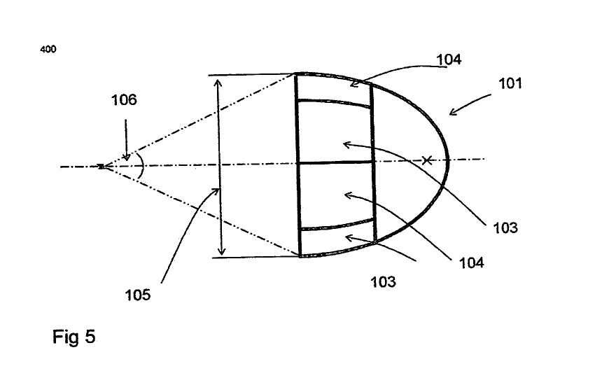

FIG. 5 shows a side view of the same reflector 101. The diameter of the reflector edge 105 is defined by the stationary reflector elements 103. It remains unaffected by movement of the movable reflector elements 104, so that the aperture angle 106 is kept constant despite any change in the reflection surface not depicted in the drawing.

1. (Cancelled)

2. The apparatus of claim 18, wherein the pressure pulse source and the reflection surface are located in a housing which is sealed on one side with the pressure pulse permeable diaphragm.

3. The apparatus of claim 2, wherein the pressure pulse source and/or at least one reflector element and/or parts thereof and/or the diaphragm surface are movable in relation to the housing.

4. The apparatus of claim 18, wherein the apparatus comprises at least two reflector elements, of which at least one is movable relative to the other and/or relative to the pressure pulse source and/or relative to the diaphragm.

5. The apparatus of claim 19,
wherein each of the reflector elements has at least one inner surface, wherein each of said inner surfaces has a reflection surface that is rotationally symmetrical relative to an exit axis and that at least one of said reflector elements is movable in an axial direction alone the exit axis.

6. The apparatus of claim 20, wherein
the inner surfaces of the reflector elements can be connected to a rotationally symmetric reflection surface relative to the exit axis; and,
part of the reflector elements can turn on bearings, so that the reflector elements belonging to this part are hinged for moving into and out of the radiation path of the pressure pulse.

7. The apparatus of claim 20, wherein
the inner surfaces of the reflector elements can be connected to a rotationally symmetric reflection surface relative to the exit axis and at least one of the reflector elements can rotate on the exit axis, so that said at least one reflector element can be moved parallel to at least one other reflector element, which remains stationary.

8. The apparatus of claim 5, wherein the movement of the movable reflector elements can be achieved manually, electrically, hydraulically or pneumatically.

9. The apparatus of claim 19, wherein the reflector elements making up the radiation path reflection surface have different reflection coefficients.

10. The apparatus of claim 9, wherein the reflector elements have different wall thicknesses.

11. The apparatus of claim 9, wherein the reflector elements have different surface roughnesses.

12. The apparatus of claim 9, wherein wherein the reflector elements are made of different materials.

13. The apparatus of claim 19, wherein the reflector elements connect to form one radiation path reflection surface, which focuses the shockwaves on a focal volume element located outside of the apparatus.

14. The apparatus of claim 13, wherein a measuring means is attached to the apparatus that indicates where the focal volume element is located.

15. The apparatus of claim 19, wherein means are attached to the apparatus that indicate the position of the reflector elements.

16. The apparatus of claim 19, wherein means are attached to the apparatus that determine the position of the reflector elements.

17. The apparatus of claim 16, wherein the measured values are recorded by a control or analysis unit.

18. An apparatus for generating shockwaves comprising:
a pressure pulse source for generating a primary pressure pulse, said primary pressure pulse having a radiation path;
at least one reflector element;
a radiation path reflection surface being located in said radiation path, wherein said radiation path reflection surface can have at least a first and a second size;
a shockwave permeable diaphragm;
a shockwave conducting medium; and
an adjustment mechanism for changing said radiation path reflection surface from said first size to said second size.

19. The apparatus of claim 18, wherein said apparatus has at least two reflector elements.

20. The apparatus of claim 19, wherein each of the reflector elements has at least one inner surface, wherein each of said inner surfaces has a reflection surface that is rotationally symmetrical relative to an exit axis.

21. A method for generating a changeable shockwave profile in a treatment area comprising:
generating a primary pressure pulse having a radiation path;
providing a reflection surface in said radiation path, wherein said reflection surface can have at least a first and a second size; and,
adjusting said reflection surface from said first size to said second size to change the shockwave profile in said treatment area.
